(19) United States
(12) Patent Application Publication
(10) Pub. No.: US 2017/0106203 A1
(43) Pub. Date: Apr. 20, 2017

(54) CONTROL OF SPIKE-TIMING DEPENDENT BRAIN NETWORK PLASTICITY VIA MULTI-COIL TRANSCRANIAL MAGNETIC STIMULATION

(71) Applicant: RIO GRANDE NEUROSCENCES, INC., Santa Fe, NM (US)

(72) Inventors: M. Bret SCHNEIDER, Portola Valley, CA (US); Amit ETKIN, Los Altos, CA (US)

(21) Appl. No.: 15/311,526

(22) PCT Filed: Jun. 1, 2015
(86) PCT No.: PCT/US2015/033557

§ 371 Date: Nov. 16, 2016

Related U.S. Application Data
(60) Provisional application No. 62/005,903, filed on May 30, 2014.

(51) Int. Cl. A61N 2/00; A61N 2/02

(52) U.S. Cl. CPC A61N 2/006; A61N 2/02

(57) ABSTRACT

Brain stimulation methods and devices in which at least two separate magnetic pulse sources with interposed space between them are placed over two distinct brain regions. The coils are pulsed at between 1 and 100 milliseconds apart (e.g., between 5 ms and 40 ms), thereby producing neuroplastic effects upon a third brain region that is network-connected to said first and second regions.

### CROSS REFERENCE TO RELATED APPLICATIONS

This patent application claims priority to U.S. provisional patent application No. 62/005,903, filed on May 30, 2014, and titled “CONTROL OF SPIKE-TIMING DEPENDENT BRAIN NETWORK PLACTICITY VIA MULTI-COIL TRANSCRANIAL MAGNETIC STIMULATION.” This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are Transcranial Magnetic Stimulation (TMS) systems and methods. In particular, described herein are TMS systems and methods for evoking changes in neural activity resembling long-term potentiation or long-term depression (plasticity) in a first region of a neural network by controlling the positioning firing times for two or more TMS electromagnets (coils) directed at spatially separated second (i.e. in addition to the first target) and/or third regions (i.e. two regions not including the first region) of a neural network in humans.

### BACKGROUND

Associative plasticity is a biological process that adjusts the strength of connections between neurons and neural networks that connect different regions of the brain. Spike-timing-dependent plasticity (STDP) is one example of associative plasticity. This process adjusts the connection strengths based on the relative timing of a particular neuron's output and input action potentials (or spikes). Among other things, STDP likely governs the development of an individual's brain, especially with regards to long-term potentiation (LTP) and long-term depression (LTD). In STDP, if an electrical input to a neuron occurs immediately before the electrical output spike of that neuron, then that particular input is made stronger. If an input spike occurs, on average, immediately after an output spike from that neuron or group of neurons, then that particular input is made somewhat weaker. Thus, inputs that might be the cause of the post-synaptic neuron's excitation are made even more likely to contribute in the future, whereas inputs that are not the cause of the post-synaptic spike are made less likely to contribute in the future. Other forms of associative plasticity use the concepts of STDP but may either not involve direct knowledge that spikes have been induced or may involve the coincident timing of two inputs where one is through a behavior or task performed rather than an electromagnetic input.

STDP has been demonstrated in animal models, including work by Henry Markram in brain slices of non-human experimental animals with dual patch clamping techniques to repetitively activate pre-synaptic neurons 10 milliseconds before activating the post-synaptic target neurons, which increased the strength of the synapse. When the activation order was reversed so that the pre-synaptic neuron was activated 10 milliseconds after its post-synaptic target neuron, the strength of the pre-to-post synaptic connection decreased. Further non-human experimental animal brain slice work by Guoqiang Bi, Li Zhang, and Huizhong Tao in Mu-Ming Poo's lab in 1998 (see the Bi GQ et al. reference listed below) showed that that synapses that are activated within 5-40 ms before a postsynaptic spike are strengthened, and those that are activated within a similar time window after the spike are weakened. STDP-related observations regarding the behavior of individual neurons in these paradigms is generally deemed to apply to groups of neurons in different but network- connected regions of the brain.

Although STDP has been applied to control of motor systems, it has not been effectively applied to higher cognitive functions using transcranial magnetic stimulation (TMS). For example, U.S. 2009/0234243 “Robotic Apparatus for Targeting and Producing Deep, Focused Transcranial Magnetic Stimulation” and U.S. 2012/0016177 “Trajectory-Based Deep-Brain Stereotactic Transcranial Magnetic Stimulation” describe rapid successive firing of TMS coils from different angles about the head. U.S. Pat. No. 7,520,848 provides a detailed treatment of how multiple channels of pulse generators are independently controlled, and thus may either generate their pulses lockstep with one another, or at separate times and rates. U.S. 2010/0256438 “Firing Patterns for Deep Brain Transcranial Magnetic Stimulation” specifies methods by which multiple coils may be pulsed at different rates and time intervals, with separate effects to areas near the coils, and to areas where the combined effects of two or more coils predominate. U.S. Pat. No. 1,316,9967 “Enhanced Spatial Summation for Deep-Brain Transcranial Magnetic Stimulation” describes the stimulation of two or more separate brain areas that are each network-connected a third brain area so as to cause changes in the activity of that third area.

U.S. Pat. No. 5,738,625, Method of and Apparatus for Magnetically Stimulating Neural Cells, involves a first and second energy source that serve to directly affect the same neuron. The first source provides a “conditioning stimulus” that serves to raise or lower the firing threshold in response to the second stimulus. This technology is not a network-based intervention nor induce lasting LTP or LTD-like plasticity.

In the Roth et al. 2014 reference (“Safety and Characterization of a Novel Multi-channel TMS Stimulator”) listed below, magnetic coil elements are physically connected and adjacent to one another, permitting no interposed spaced between first and second magnets. This imposes the limitation that the first and second areas that are stimulated must be directly adjacent to one another. Like other studies before it, this work is focused on short-interval cortical inhibition and facilitation, wherein a conditioning pulse is applied to a target region (typically at 80% of motor threshold) and then a test pulse is applied to a region that provides input to the target region (typically at 120% of motor threshold). The interval between these pulses is very short (1-5 ms typically) and capitalizes on local, non-placticity-based after-effects that occur immediately following a TMS pulse, and which gate the effects of incoming signal. This approach does not induce lasting LTP or LTD-like plasticity.

STDP holds the potential for finding rTMS treatments that are more effective at modulating brain activity at targeted locations, make those therapeutic effects more durable, or might be able to do so in a far shorter amount of treatment time.

### REFERENCES

U.S. 2009/0234243 “Robotic Apparatus for Targeting and Producing Deep, Focused Transcranial Magnetic Stimulation” Schneider M B, Mishelevich DJ.

U.S. 2012/0016177 “Trajectory-Based Deep-Brain Stereotactic Transcranial Magnetic Stimulation” Mishelevich D J, Schneider M B

PCT/US2008/073751 Firing Patterns for Deep Brain Transcranial Magnetic Stimulation. Mishelevich D J, Schneider M B.

U.S. application Ser. No. 13/169,967. Schneider M B. Enhanced Spatial Summation for Deep-Brain Transcranial Magnetic Stimulation

U.S. Pat. No. 5,738,625. Method of and Apparatus for Magnetically Stimulating Neural Cells. Gluck D S.

Roth Y, Levkovitz Y, Pell G S, Ankry M, Zangen A. Safety and Characterization of a Novel Multi-channel TMS Stimulator. Brain Stimul. 2014 March-April; 7(2):194-205. doi: 10.1016/j.brs.2013.09.004. Epub 2013 Dec 10.

Levy W B, Steward O (April 1983). “Temporal contiguity requirements for long-term associative potentiation/depression in the hippocampus”. Neuroscience 8 (4): 791-7.doi:10.1016/0306-4522(83)90010-6. PMID 6306504.

Dan Y, Poo M M (1992). “Hebbian depression of isolated neuromuscular synapses in vitro”. Science 256 (5063): 1570-73. PMID 1317971.

Debanne D, Gähwiler B, Thompson S (1994). “Asynchronous pre- and postsynaptic activity induces associative long-term depression in area CA1 of the rat hippocampus in vitro.”. PNAS 91 (3): 1148-52. doi:10.1073/pnas.91.3.1148. PMC 521471. PMID 7905631.

Markram H, Lübke J, Frotscher M, Sakmann B (January 1997). “Regulation of synaptic efficacy by coincidence of postsynaptic APs and EPSPs”. Science 275 (5297): 213-5.doi:10.1126/science.275.5297.213. PMID 8985014.

Bi G Q, Poo M M (15 Dec. 1998). “Synaptic modifications in cultured hippocampal neurons: dependence on spike timing, synaptic strength, and postsynaptic cell type”. J. Neurosci. 18 (24): 10464-72. PMID 9852584XXX

### SUMMARY OF THE DISCLOSURE

In general, described herein are methods and devices for evoking plasticity of a neural network (e.g., in a brain) by non-invasive transcranial magnetic stimulation (TMS).

This evoked plasticity may be referred to as associative plasticity, which may be due to spike-timing-dependent plasticity (STDP). For convenience, the plasticity effects described herein are referred to as STDP (or STDP-like) since it exhibits the characteristics of STDP. The effects and methods described herein may be referred to generally as associative plasticity, as it is not feasible to record spikes in a living human nervous system, which may require invasive and damaging techniques.

For example, described herein are methods of evoking long-term potentiation/plasticity of a first region of a patient's brain by directing a first TMS stimulation protocol to a second brain region and directing a second TMS stimulation protocol to drive stimulation at a third brain region, within a predetermined time period, such as between 5 ms and 40 ms, or more specifically, between 10 ms and 40 ms, between 10 ms and 30 ms, etc. The second stimulation protocol may be triggered between 5 ms and 40 ms after stopping the application of the first TMS stimulation protocol. The first, second and third brain regions may be separate brain regions, though they may be connected as part of a network (e.g., first-order connections, second-order connections, third-order connections, etc.). In some variations the first and second (or first and third) brain regions are the same. The specific time windows quoted here are not fixed but rather are relative to the neural regions and connections affected, and may be furthermore altered by behavioral states an individual is in or medications given.

For example, described herein are methods and devices by which magnetic coils located over second and third distinct brain regions, and are discharged in rapid succession so as to elicit neuroplasticity in the first and second or a third brain region that is network-connected to the first two regions. The time between pulses of the stimulation protocols to the two coils over the two brain regions is generally in the range 5 to 40 ms, and the stimulation protocols are timed to occur either before or after discharge within the third brain region.

### BRIEF DESCRIPTION OF THE DRAWINGS

FIG. 1 shows and embodiment in which coils are located over separate regions of the frontal lobe (dorsomedial prefrontal cortex and dorsolateral prefrontal cortex, respectively), with space separating the stimulating coils. A third region (anterior cingulate cortex) is modulated.

FIG. 2 shows an alternative embodiment in which Coil A and Coil B are over opposite brain hemispheres, each of which is network-connected with third region, the anterior cingulate cortex.

FIG. 3 shows two non-adjacent stimulation coils over two distinct brain regions: the left dorsolateral prefrontal cortex and the midline bilateral dorsomedial prefrontal cortex. Stimulation at these sites can be anticipated to modulate sites of primary stimulation, as well as connected areas including the dorsal anterior cingulate.

FIG. 4 shows a simplified schematic of a general STDP paradigm.

FIG. 5 is an example of a time table for firing two coils and two respective locations.

### DETAILED DESCRIPTION

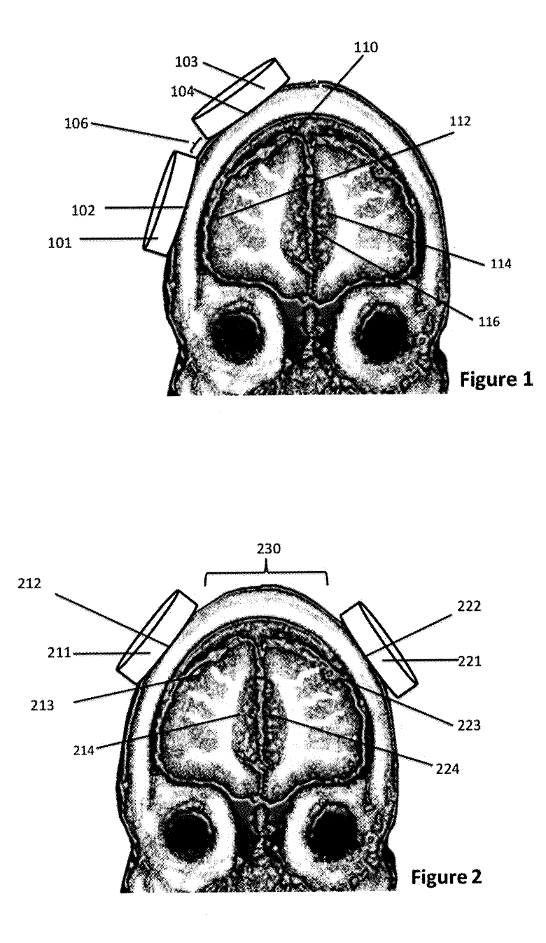

FIG. 1 shows and embodiment in which Coil A 101 and Coil B 103 are located over separate regions of the frontal lobe, e.g., dorsomedial prefrontal cortex (dmPFC) 110 and dorsolateral prefrontal cortex (DLPFC) 112, respectively, with space 106 separating the stimulating coils. The 3rd regions right anterior cingulate cortex (ACC) 114 and left anterior cingulate 116 are thereby modulated via internal network connectivity. The optimal time between pulses to the two coils is generally in the range 5 to 40 ms either before or after stimulation to the reference neuron. A region of the scalp 102 is interposed between coil A 101 and the dorsolateral prefrontal cortex surface 112, and a second region of the scalp 104 is interposed between the dorsomedial prefrontal cortex 110 and coil B 103. A space 106 separates the stimulating coils 101 and 103.

FIG. 2 shows an alternative arrangement of coils in which Coil A 211 and Coil B 221 are over opposite brain hemispheres, each of which is network-connected with third region, e.g., the right anterior cingulate 214 and third region left anterior cingulate 224. Scalp under Coil A 212 overlies the left brain surface under Coil A 213, and Scalp under coil B 222 overlies the right brain surface region 223. Between the coils at the surface of the scalp lies space between stimulating coils 230. Additionally, bilateral DLPFC stimulation may serve to modulate ventromedial PFC and ventral ACC.

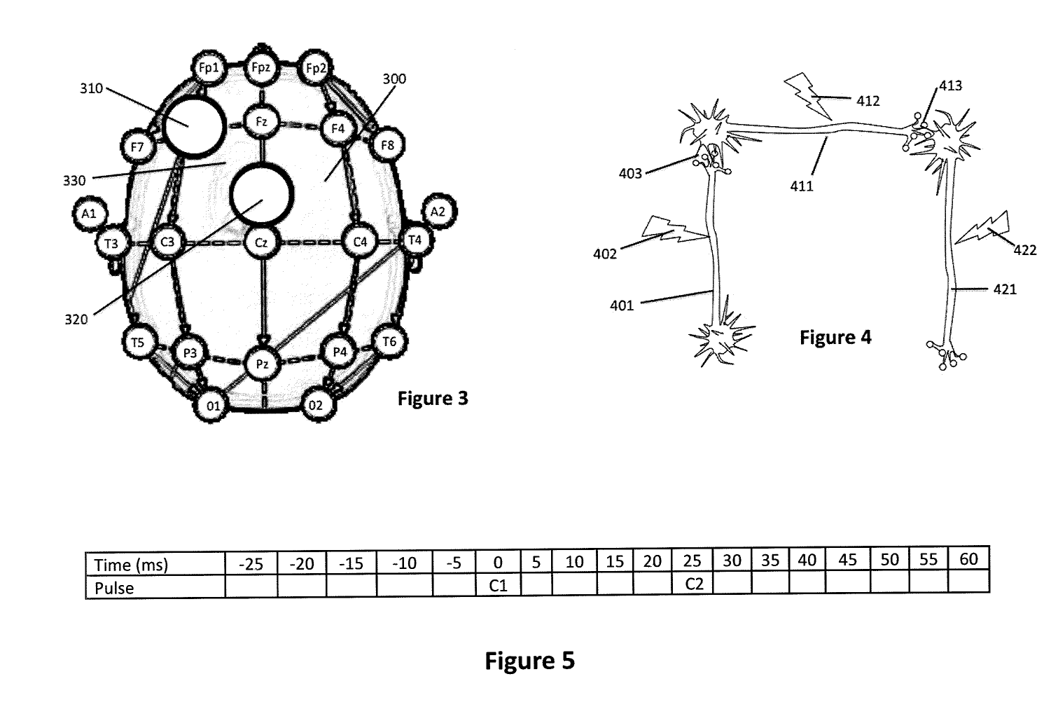

FIG. 3 shows two non-adjacent stimulation coils, Coil A 310, and Coil B 320, separated by a space 330, shown in the context of an EEG 10-20 map of the scalp surface 300. Coil A 310 is positioned over the left dorsolateral prefrontal cortex and Coil B 320 is positioned over the midline bilateral dorsomedial prefrontal cortex. Stimulation at these sites can be anticipated to modulate sites of primary stimulation, as well as connected areas including the dorsal ACC, ventral ACC and ventromedial prefrontal cortex.

Many different brain networks can be targeted with the device and method as described. In an alternative embodiment, both coils are placed over two different parts within the DLPFC—such as two locations on one side that are part of two different brain networks: one that triggers activation in “salience monitoring regions”, like the dorsal anterior cingulate cortex (dACC) and insula, and the second that triggers activation in “task-directed attention” regions (related to the frontal-parietal “executive” network). These two networks influence each other and the executive network influences a third network, the default mode network (including the ventromedial prefrontal cortex). Associative plasticity (e.g., STDP) stimulation to each of these DLPFC targets could modify both the communication between the salience and executive networks as well as their ability to modulate the default mode network. If the salience and executive networks are hierarchically arranged, as suggested by some recent work (e.g., salience signaling to executive which signals to default mode network) then the directional aspect of STDP modulation could help either enhance or weaken this hierarchical network regulatory relationship.

In an alternative embodiment, bilateral DLPFC stimulation may be used to modulate ventromedial PFC and ventral ACC.

In an alternative embodiment the DLPFC and parietal cortex may be stimulated to enhance both DLPFC-to-parietal (top-down) and parietal-to-DLPFC (bottom-up) interactions.

In an alternative embodiment, the DLPFC and frontopolar cortex may be stimulated as means to reach third region vmPFC and thereby the brain's default mode network (DMN)

In an alternative embodiment DLPFC and cerebellum may be stimulated as means to reach third region fronto-striatal cognitive circuitry.

Neurological disorders may be treated with the approach described herein, and include as examples the sequential stimulation of bilateral motor and premotor cortices for the treatment of Parkinson's disease. Sequential stimulation of Wernicke's and Broca's areas may be useful for the treatment of stroke leading aphasia.

FIG. 4 shows a simplified schematic of a general STDP paradigm. This example may involve stimulating two separate areas of the cortical surface that have tracts that lead to the deep medial prefrontal cortex.

Neuron A 401 is stimulated by electromagnetic pulse 402, thereby affecting Neuron A/Neuron B coupling 403; Neuron B 411 is stimulated by electromagnetic pulse 412 thereby affecting Neuron/Neuron C coupling 413. Neuron 421 is stimulated by Electromagnetic pulse 422 and so on.

If action potential at B occurs before action potential at C, then B/C coupling becomes functionally stronger, resulting in a larger input to C from output of B. If action potential at B occurs after action potential at C, then the B/C coupling becomes functionally weaker, resulting in smaller input to C from output of B.

FIG. 5 is an example of a timetable for firing two coils and two respective locations. Coil #1 “C1” is fired at t=0 ms, while Coil #2 “C2 is fired 25 ms later at t=25 ms. Of course, this is one hypothetical example, and it will be appreciated that any permutation within such a table is anticipated. In any case, a time interval separates the firing of two or more coils, and the resultant activity changes primary areas stimulated, and in third areas of network influence may be documented by means including EEG, PET and fMRI.

The invention described herein is significantly different from what has been shown or suggested by the prior art. For example, as distinct from recent publications on multi-coil TMS arrays, and the previously characterized more wide-spread use of two independently controlled separate TMS coils, the result (and goal) here is to engage the brain's endogenous plasticity mechanisms to create long-term plasticity. Prior studies have focused on and have only achieved short-interval cortical inhibition and facilitation, wherein a conditioning pulse is applied to a target region (typically at 80% of motor threshold) and then a test pulse is applied to a region that provides input to the target region (typically at 120% of motor threshold). The interval between these pulses is very short (typically <1-5 ms) and capitalizes on local after-effects that occur immediately following a TMS pulse, and which gate the effects of an incoming neural signal. This type of inhibition or facilitation is distinct from the long-lasting synaptic plasticity that is the mechanism of STDP, which rather relates to how the brain encodes information normally through associative learning. The intervals between two TMS pulses meant to induce associative plasticity as described herein (e.g., STDP) are typically 10-40 ms and will engage distinct cellular and circuit-level processes compared to the short-interval methods described above. Additionally, repetition of STDP stimulation will then produce long lasting effects that outlast the stimulation itself, which is not the case for short-interval inhibition/facilitation. Finally, STDP has been published in the past by activation of ascending sensory input into sensory/motor cortex by stimulation of a peripheral nerve, which serves to provide the input to this region, which is subsequently activated with a TMS pulse. This induces STDP but is an approach distinct from what is described herein, wherein two brain regions are targeted and STDP-like associative plasticity is achieved by coordinating their activation.

The STDP-like associative plasticity described herein is also inherently directional (speaking to which neuron or brain region is activated before versus after another). Accordingly, the sequence of TMS coil firing will determine the specific pathway in the brain that undergoes plasticity and the direction of the effect. In other words if we want to potentiate or depress the pathway from region A to B then we fire a TMS coil over A and then the TMS coil over B. If we want to potentiate the reverse pathway (B to A) then we reverse the order or stimulation. Inasmuch as these regions are reciprocally connected, there may be a convergence between potentiation versus depression (determined by the sequence of firing) and the direction of effect (A to B and B to A). However, if there are specific pathways in the brain connecting regions in a more hierarchical manner (i.e. only A to B, as expected for many neural systems) then this will further constrain the potential effects observed. When considering deep or downstream effects (e.g. on region C), then firing A and then B would facilitate the B to C pathway while B then A would facilitate the A to C pathway. As detailed in the description of FIG. 4, the presynaptic/postsynaptic direction determines in how spike timing will affected the circuit. Therefore, depending upon the experimental or therapeutic goals, the labels “Coil A” and “Coil B” and their associated discharge timing may be interchanged from those illustrated in the figure. As a specific illustration of this, by reversing the order of stimulation in the setup previously described, the DLPFC-to-ACC interaction becomes an ACC-to-DLPFC interaction. The directional effect may also be reversed if alterations are made to coil orientation, pharmacological or behavioral states, or other additional intervention that can result in an “anti-Hebbian” process by which firing a coil over A then over B causes B to A plasticity rather than the expected “Hebbian” process causing A to B plasticity.

Also described herein are methods and apparatuses for strengthening or weakening a connection between two brain regions (generically, region A and region B of a subject's brain). For example, the STDP-like effects described herein may be used to strengthen or weaken the connection between region (or neuron) A and region (or neuron) B, depending on the sequence of activation of each region. For example, response inhibition may be modulated by STDP between two brain regions such as the inferior frontal gyrus (IFG) and the anterior portion of the supplementary motor area (pre-SMA). A first TMS coil may be placed over the IFG and another coil over the pre-SMA and stimulating as described herein. For example, applying power to the coils to TMS sequentially to the regions with a delay of between about 5 ms and about 40 ms between stimulation of each region, so that a neuroplastic effect is achieved.

In addition the TMS stimulation, any of the methods described herein may be paired with non-TMS stimulation to evoke brain activity that may be paired in the timed manner described herein to create associative plasticity. Thus, any of the methods described herein may include a combination of TMS (e.g., stimulation by one or more TMS coils) and a behavior or other stimuli that elicits brain activity. The TMS and the behavior or other stimuli may be timed as described herein; for example, having a delay of between 5 ms and 40 ms (e.g., between 10 ms and 40 ms). In one example, a non-TMS stimuli that can be applied with TMS to induce associative plasticity is a fear-conditioned stimulus that may, for example, cause a subject's amygdala to increase its firing rate, and this fear-conditioned stimulus may be timed with TMS pulses to the medial or lateral prefrontal cortex to create STDP targeting the amygdala.

Thus, for example, a method of inducing long term potentiation in a target brain region may include providing a non-TMS stimulation (e.g., a sensory input, such as a visual input, audible input, tactile input, etc., including combinations thereof) that evokes spiking (neuronal firing) in a first brain region, and applying (within a predetermined time period, e.g., between about 5 ms and 40 ms (e.g., between 10 ms and 40 ms, etc.) a TMS stimulation. Pairing the stimulation in this manner may result in an STDP-like effect.

Any of the variations described herein may also alternatively or additionally include giving a drug or having the person engage in a behavior as part of the method. The drug may be a drug that modulates neuronal excitability, particularly drugs that modulate regions of the brain that are being targeted by the method (for example, drugs such as isoflurane are known to modulate neuronal excitability of the nucleus reticularis thalami).

As used herein the steps of applying the first and second stimulation (e.g., TMS stimulation) may be paired such that the first stimulation is completed before starting the second stimulation, e.g., within the window of time, such as 5 ms to 40 ms (10 ms to 40 ms, etc.) for inducing the STDP-like effect described herein. Thus, the first stimulation (e.g., TMS stimulation, non-TMS stimulation), may be ‘off’ for at least 5 ms (e.g., between 5 ms and 39 ms) prior to starting the second, e.g., TMS stimulation. In some variations, the second stimulation may be completed before the window of time (e.g., 5 ms to 40 ms from the end of the first stimulus) has closed; in some variations the second stimulation may last for a period after the first stimulation has ended.

Any of the methods described herein may be applied to induce anti-Hebbian plasticity in some variations. In this case, LTP/LTD may be elicited using the reverse of pairing sequence described above. This may be achieved, in some variations, by reversing the coil orientation. In some variations rather than stimulating the first target region with a first stimulation (e.g., TMS), before the connected (e.g., first- or second-order connected) second target region, the second target region may be stimulated before the first target region.

In any of the methods and apparatuses described herein, the TMS stimulation may be provided by multiple TMS coils targeting the different brain regions. For example, a plurality of TMS coils may target the first region, and one or more TMS coils may target the second region. In such case, the use of multiple coils targeting the same region may provide deeper target-specific brain stimulation that is otherwise possible with a single TMS coil.

As mentioned above, the methods described herein may be used to regulate activity of a brain region by targeting two other (distinct, but connected) brain regions by applying STDP-like stimulation as described above.

In any of the methods described herein, the method may be used to modulate brain function by strengthening or weakening communication between two or more distinct cognitive networks in the brain, not limited to (or excluding) motor control networks. Because these networks may not be localized (but may be distributed through the brain or brain regions), targeting may be important. In particular it may be beneficial to target the portion of each network that corresponds to the ‘control’ point (or points) of the network. As used herein a control point of a network is a portion of the network that is causally linked to the regulation of the network. A control point of a network may be identified for a part of a network by empirically determining what targets in the network exert an effect (different from sham) when stimulation is applied. Thus, in any of the methods described herein, the method may include a step or steps to identify the control point(s) of the target or target network, and apply TMS to the identified control points. In some variations, the control point(s) may be known from the literature (see, e.g., Chen et al, “Causal interactions between fronto-parietal central executive and default-mode networks in humans”, Proc Natl Acad Sci USA. 2013 Dec. 3; 110(49): 19944-19949.). In this example, the communication between the fronto-parietal central executive and default-mode networks by specifically targeting control points in each network and applying TMS with one or more coils aimed specifically at the control regions for each network.

These method may also be applied to control points by first empirically determining the location of the control point, as mentioned. For example, prior to the application of STDP-like stimulation as described above, functional imaging (e.g., fMRI) and targeted TMS may be used in combination to identify the control point(s) of the network. Thus, a control point that couples the stimulation by TMS with evidence from neuroimaging showing a downstream (e.g., network-induced) effect because of the TMS can be used to identify and target the later coordinated STDP-like stimulation of each control point (or control region) with a delay of between 5 and 40 ms (or as otherwise appropriate) to strengthen or weaken communication between the different networks, or a third network in communication with one or both of these.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of inducing long term potentiation in a target brain region, the method comprising:
positioning at least two separate magnetic pulse sources over two distinct brain regions, so that there is a space between said magnetic field sources;
applying power to pulse the magnetic pulse sources sequentially with a delay of between about 5 ms and about 40 ms delay between stimulation of each magnetic pulse source, so that a neuroplastic effect is achieved upon a third brain region that is network-connected to said first and second regions.

2. The method of claim 1, wherein positioning comprises positioning the at least two separate magnetic pulse sources to target control points within each of the two distinct brain regions so that the control points receive more energy than non-control points within the brain regions.

3. The method of claim 2, further comprising identifying the control points for the first and second region.

4. The method of claim 3, wherein identifying comprises using both fMRI and TMS to identify the control points.

5. The method of claim 2, wherein the first and second region comprise networks.

6. The method of claim 1, wherein the at least two separate magnetic pulse sources comprises a first TMS electromagnet and a second TMS electromagnet.

7. The method of claim 1, wherein positioning comprises positioning a first TMS electromagnet over a dorsomedial prefrontal cortex and positioning a second TMS electromagnet over a dorsolateral prefrontal cortex.

8. The method of claim 7, wherein applying power comprises applying power so that a neuroplastic effect is achieved upon an anterior cingulate cortex and a left anterior cingulate.

9. The method of claim 1, wherein applying power comprises applying power to a second TMS electromagnet between about 5 ms and about 40 ms after the application of power to a first TMS electromagnet has stopped.

10. A brain stimulation device configured to evoke long-term potentiation in a brain target, the device comprising:
a first magnetic pulse source configured to be positioned over a first brain region;
a second magnetic pulse source configured to be positioned over a second brain region;
wherein the first and second pulse sources are adapted to be positioned over a subject's head with a space between the first and second pulse sources; and
a controller having a trigger, wherein the controller is configured such that activation of the trigger causes the application of a first pulse regime by the first magnetic pulse source followed by a preset delay of between about 1 ms and about 100 ms before the application of a second pulse regime by the second magnetic pulse source.

11. The device of claim 7, wherein the first and second magnetic pulse sources comprise TMS electromagnets.

12. The device of claim 7, wherein the first and second magnetic pulse sources are independently positionable.

13. The device of claim 7, wherein the controller is configured so that the activation of the trigger causes the application of the first pulse regime by the first magnetic pulse source followed by a preset delay of between about 5 ms and about 40 ms after the stop of the first pulse regime before the application of the second pulse regime by the second magnetic pulse source.

14. A method of non-invasively stimulating the brain to change a subject's behavior, and/or ameliorate a neurological or psychiatric disorder, the method comprising:
positioning a first TMS magnetic coil over a first brain region and a second TMS magnetic coil over a second brain region, wherein the first and second brain regions are connected within a neural network and wherein the first and second brain regions are separated by an intervening brain region;
applying stimulation from the first TMS magnetic coil to the first brain region, waiting for a waiting period of between about 5 and about 40 ms and then applying stimulation from the second TMS magnetic coil to the second brain region, wherein the intervening brain region receives stimulation during the application of the first stimulation and the second stimulation at levels below that of areas beneath said first and second coils;
whereby an action potential occurs within the waiting period in the second brain region, producing neuroplastic changes in the relationship between the first and second brain region.

15. An apparatus for non-invasively stimulating the brain to change a subject's behavior, and/or ameliorate a neurological or psychiatric disorder, the apparatus comprising:
a first pulsed magnetic field delivery device adapted to be non-invasively placed over a first scalp region;
a second pulsed magnetic field delivery device adapted to be non-invasively placed over a second scalp region, wherein the first and second scalp regions are non-contiguous;
a controller configured to drive pulsing of the first and second pulsed magnetic field delivery devices with a pre-specified timed interval between the completion of activation of the first pulsed magnetic field delivery device and the start of activation of the second pulsed magnetic field delivery device, whereby an action potential occurs within said pre-specified time interval in a region of the brain under the second scalp region, producing neuroplastic changes in the relationship between a first brain region under the first scalp region and the second brain region under the second scalp region.
